(19) United States
(12) Patent Application Publication
(10) Pub. No.: US 2007/0178131 A1
(43) Pub. Date: Aug. 2, 2007

(54) Medical device for insertion into human body, and method of manufacture

(75) Inventors: Hiroshi Yamada, Otsu-shi (JP); Chisaka Aoyama, Otsu-shi (JP); Kae Fujiwara, Otsu-shi (JP); Kenji Hioki, Otsu-shi (JP)

Correspondence Address: GLOBAL IP COUNSELORS, LLP, 1233 20TH STREET, NW, SUITE 700, WASHINGTON, DC, 20036-2680, US

(73) Assignee: I.S.T. Corporation, Otsu-shi (JP)

(21) Appl. No.: 11/436,053

(22) Filed: May 18, 2006

(30) Foreign Application Priority Data
Feb. 2, 2006 (JP) 2006-026125

(51) Int. Cl. A61F 2/02; A61M 31/00

(52) U.S. Cl. 424/423; 604/500

(57) ABSTRACT

A medical device for insertion into the human body has improved sliding characteristics compared to similar devices with fluororesin surfaces. The medical device for insertion into the human body includes a substrate having given shape, and an agent for improving the sliding characteristics having a hydrophilic block and a hydrophobic block, and coating the hydrophobic surface.

### TECHNICAL FIELD

The present invention relates to a medical device for insertion into the human body such as a medical catheter, medical wire or the like and a method for its manufacture.

### Background Art

It has been reported in the past that, “it is possible to increase sliding characteristics by coating the surface of a medical device for insertion into the human body such as a medical catheter, medical wire or the like with a fluororesin” (for example, see patent document 1).

[Patent Document 1]

Japanese published unexamined application No. 2005-205183

[Invention Disclosure]

### Problem to be Solved by the Invention

However, in the industrial arena in recent years, there has been a demand for a further increase in sliding characteristics in such a medical device for insertion into the human body.

An object of the present invention is to provide a medical device for insertion into the human body that possesses sliding characteristics that are superior to those in medical devices for insertion into the human body that possess fluororesin surfaces, and to provide a method for manufacturing the medical devices.

### Means to Solve the Problem

From the results of repeated efforts with experiments and inquiries, applicants have discovered a medical device for insertion into the human body that possesses superior sliding characteristics compared to medical devices for insertion into the human body that possess fluororesin surfaces. The device comprises a substrate and an agent for improving the sliding characteristics. The substrate has a given shape and a hydrophobic surface on at least a portion. The agent for improving the sliding characteristics has a hydrophilic block and a hydrophobic block and coats the hydrophobic surface. Furthermore, examples of what is referred to here as a “hydrophobic surface” can include a fluororesin surface, olefin resin surface, ester resin surface, acrylate resin surface, polyurethane resin surface, polyamide resin surface and the like. Moreover, examples of what is referred to here as a “fluororesin” can include poly(tetrafluoroethylene) (PTFE), tetrafluoroethylene-perfluoroalkylvinyl ether copolymer (PFA), poly(chlorotrifluoroethylene) (PCTFE), tetrafluoroethylene-hexafluoropropylene copolymer (FEP), tetrafluoroethylene-ethylene copolymer (PETFE) and the like, or blends of the foregoing. In addition, examples of what is referred to here as an “olefin resin” can include polyethylene, polypropylene, polybutadiene, poly(vinyl chloride) and the like, or blends of the foregoing. Additionally, examples of what is referred to here as an “ester resin” can include ethylene-vinyl acetate copolymer, poly(ethylene terephthalate), poly(butylene terephthalate) and the like, or blends of the foregoing. Moreover, examples of what is referred to here as an “acrylate resin” can include poly(acrylic acid), poly(methyl methacrylate) and the like, or blends of the foregoing. In addition, examples of what is referred to here as a “polyamide resin” can include Nylon 6, Nylon 66, Nylon 12 and the like, or blends of the foregoing.

Additionally, in the present invention, the hydrophobic surface is preferably a fluororesin surface.

Moreover, in the present invention, the agent for improving the sliding characteristics preferably has a thickness of 0.1-2 μm.

In addition, in the present invention, the hydrophilic block is preferably a polyoxyethylene group.

Additionally, in the present invention, the number of the repeating units of the polyoxyethylene group preferably includes 1-5.

Moreover, in the present invention, the hydrophobic block is preferably a hydrocarbon group. Furthermore, examples of what is referred to here as a “hydrocarbon group” that can be mentioned include alkyl groups, alkenyl groups, alkynyl groups and the like. In addition, the hydrocarbon group can also contain heteroatoms.

Additionally, in the present invention, it is preferable for the hydrocarbon group to be selected from the group consisting of oleyl group, lauryl group and stearyl group.

Moreover, in the present invention, preferred agents for improving the sliding characteristics (so-called neutral surfactants (nonionic surfactants)) are as shown in preferred by generic formula (1) below:
R¹O(CH₂CH₂O)ₙH (1)
(wherein, R¹ is a C8-C22 hydrocarbon group and n is 1-5), or by generic formula (2) below:
R²COO(CH₂CH₂O)ₙH (2)
(provided that in the formula R² is a C7-C21 hydrocarbon group and n is 1-3), or by generic formula (3) below:
R³O(CH₂CH(CH₃)O)ₘH (3)
(wherein, R³ is a C8-C22 hydrocarbon group and m is 1-5), or by generic formula (4) below:
R⁴COO(CH₂CH(CH₃)O )ₘH (4)
(wherein, R⁴ is a C7-C21 hydrocarbon group and m is 1-3), or by generic formula (5) below:
R⁵O(CH₂CH(CH₃)O)ₛ(CH₂CH₂O)ₜH (5)
(wherein, R⁵ is a C8-C22 hydrocarbon group and s and t are 1-5), or by generic formula (6) below:
R⁶COO(CH₂CH(CH₃)O)ᵤ(CH₂CH₂O)ᵥH (6)
(wherein, R⁶ is a C7-C21 hydrocarbon group and u and v are 1-3).

In addition, in the present invention, when the hydrophobic surface is a fluororesin surface, it is preferable for the hydrophobic block to be a fluorine-containing hydrocarbon group.

Furthermore, in the present invention the substrate of the medical device for insertion into the human body is in the form of a medical wire or a medical catheter (tube) or the like.

Additionally, in the present invention, when the substrate is a catheter, the hydrophobic surface is on at least one of the inner surface and the outer surface of the catheter.

Furthermore, in the present invention, examples in addition to the agent for improving the sliding characteristics can include poly(ethylene glycol), or polycations or polyanions and the like. Moreover, examples of these polycations can include quaternized polyacrylamides, or polyvinylpyrrolidine, polyvinylamine, polyallylamine, polyethyleneimine, polyvinylvinylidene, Nafion (registered trademark) that possesses a quaternized end group, poly(dimethyldiallylammonium chloride), as well as synthetic and natural polymers of electrolytes that possess quaternized nitrogen-containing end groups. In addition, examples of these polyanions can include poly(ethylene sulfonate), poly(styrene sulfonate), Nafion, perfluoroacid ionomers and the like.

Additionally, the present applicants discovered that the above type of medical device for insertion into the human body can be manufactured according to a manufacturing method that is explained as follows. For the manufacturing method for a medical device for insertion into the human body, the medical device is manufactured with at least a step for applying the agent for improving the sliding characteristics and a drying step. In the step for applying an agent for improving the sliding characteristics, coating the substrate with the agent for improving the sliding characteristics is carried out by bringing a solution of the agent for improving the sliding characteristics into contact with the hydrophobic surface of the substrate. In the drying step, the substrate coated with the agent for improving the sliding characteristics is dried.

Furthermore, in the present invention, it is preferable for 0.1-5 wt % of the agent for improving the sliding characteristics to be contained in the solution.

Moreover, in the present invention, when the agent for improving the sliding characteristics is poly(ethylene glycol monooleate), it is preferable to maintain the solution at 60±5° C.

### EFFECT OF THE INVENTION

According to the experimental results, a medical device for insertion into the human body in accordance with the present invention will exhibit superior sliding characteristics in both the dry state and in the wet state as compared to the medical device possessing a fluororesin surface. In addition, the medical device for insertion into the human body will have superior durability, and the extent of degradation due to repeated use will be extremely small. Furthermore, the reason why the medical device for insertion into the human body according to the present invention exhibits such superior physical properties is unobvious.

Additionally, a conventional sliding surface would have a water-swellable gel film that coats a catheter or a medical wire, but such a catheter or a medical wire would not possess adequate sliding characteristics unless it had a gel film thickness of ≧50 μm. However, it was discovered that a catheter or a medical wire in accordance with the present invention that has a surface treatment agent with thickness on the order of 0.1 μm will have adequate sliding characteristics. Consequently, a catheter or a medical wire in accordance with the present invention has the characteristic that it can ensure adequate sliding characteristics even at small diameters.

Moreover, the method for manufacture of the medical device for insertion into the human body according to the present invention makes it possible to manufacture such a superior medical device for insertion into the human body.

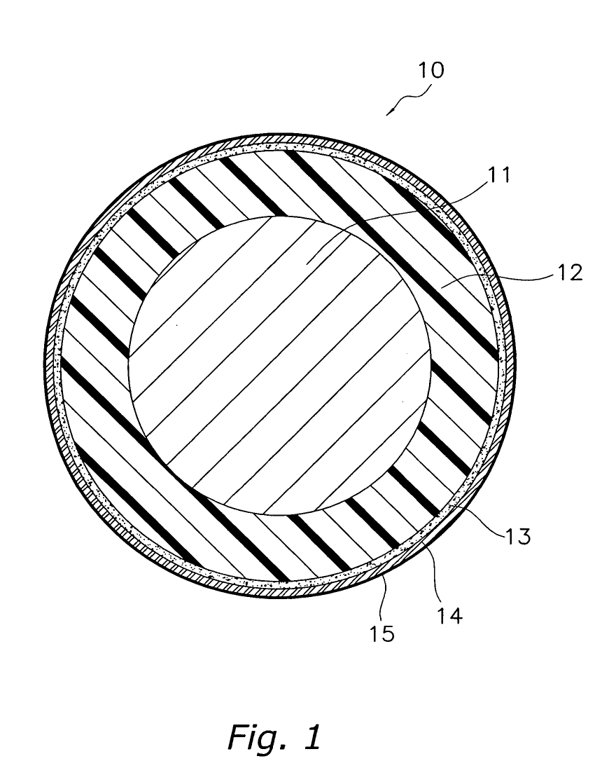

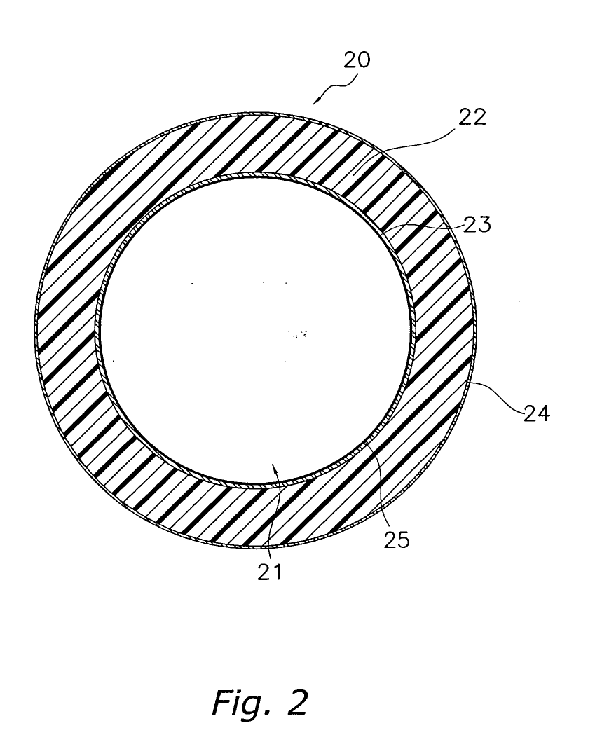

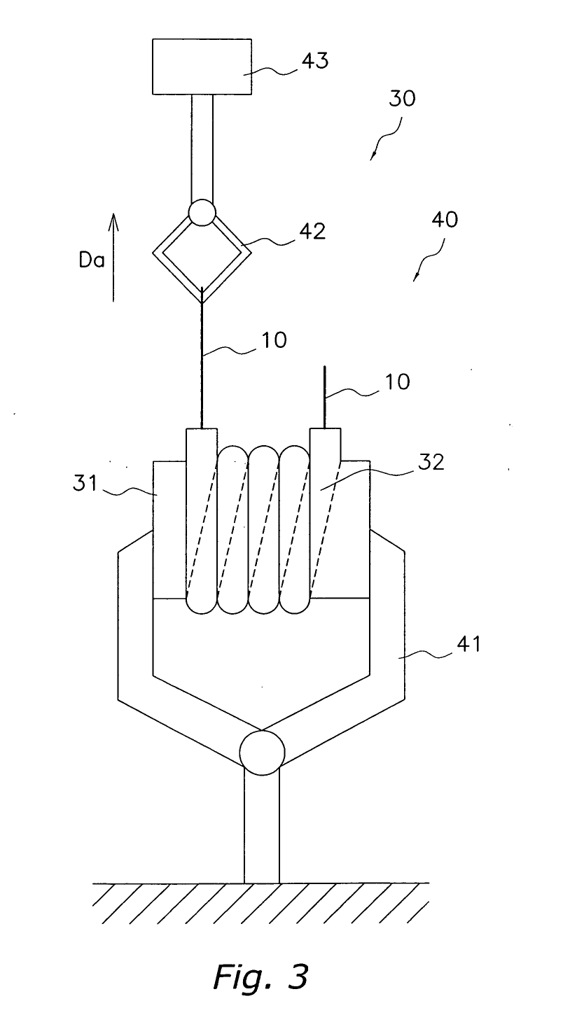

### BRIEF EXPLANATION OF DIAGRAMS

[FIG. 1] It shows a cross-sectional view of a medical wire with improved sliding characteristics in accordance with the present invention.

[FIG. 2] It shows a cross-sectional view of a medical catheter with improved sliding characteristics in accordance with the present invention.

[FIG. 3] It is a diagram showing the method for measuring the frictional resistance of the medical wire with improved sliding characteristics and the medical catheter with improved sliding characteristics in accordance with the present invention.

### EXPLANATION OF SYMBOLS

10 Medical wire with improved sliding characteristics
14 Fluororesin layer
15, 25 Improved sliding characteristics layer
20 Medical catheter with improved sliding characteristics
22 Fluororesin
   [Best Mode for Implementing the Invention]

A description is provided below of a structure and a method of for the manufacture of the medical wire with improved sliding characteristics and the medical catheter with improved sliding characteristics according to one embodiment of the present invention. Furthermore, the description of the structure and the method for the manufacture are divided into the description of the medical wire with improved sliding characteristics and the description of the medical catheter with improved sliding characteristics.

[Medical Wire with Improved Sliding Characteristics]

(1) Structure of the Medical Wire with Improved Sliding Characteristics

As shown in FIG. 1, medical wire with improved sliding characteristics 10 according to this embodiment of the present invention is provided with metal wire 11 as the core material, intermediate resin layer 12 coating the outer surface of metal wire 11, fluororesin adhering layer 13 coating the outer surface of intermediate resin layer 12, fluororesin layer 14 coating the outer surface of fluororesin adhering layer 13, and improved sliding characteristics layer 15 coating the outer surface of fluororesin layer 14.

For metal wire 11 utilized in this embodiment of the present invention, either a straight shape or a fine-pointed taper shape is preferred. In addition, this metal wire 11 is preferably formed from a superelastic alloy, stainless steel or the like. Examples of the superelastic alloy can include Ti—Ni (Ni: 49-51 atom %, including a third element added to Ti—Ni), Cu—Al—Zn (Al: 3-8 atom %; Zn: 15-28 atom %), Fe—Mn—Si (Mn: 30 atom %, Si: 5 atom %), Cu—Al—Ni (Ni: 3-5 atom %, Al: 28-29 atom %), Ni—Al (Al: 36-38 atom %), Mn—Cu (Cu: 5-35 atom %), Au—Cd (Cd: 46-50 atom %) and the like.

A preferred intermediate resin layer 12, without sacrificing the flexibility of metal wire 11, is formed from at least one synthetic resin selected from a group consisting of chemically and mechanically stable nylon, poly(vinyl chloride), polypropylene, polyamide, polyimide, silicone rubber, polyurethane and composites of the foregoing. Furthermore, in this embodiment of the present invention, in order to be able readily to confirm the location of the medical device with improved sliding characteristics that has been inserted into the human body, there is no problem adding an X-ray contrast substance such as tungsten powder or barium powder with the resin that is used to form the intermediate resin layer 12.

For fluororesin adhering layer 13, it is preferable for fluororesin powder to be contained in at least one resin selected from the group consisting of polyamide-imide resins, epoxy resins, poly(phenylene sulfide) resins, poly(ether sulfone) resins, poly(ether ketone) resins, poly(ether amide) resins, polysulfone resins and polyimide resins as well as derivatives of the foregoing. This is in order to increase the adhesiveness toward outermost fluororesin layer 14. Examples of the fluororesin can include poly(tetrafluoroethylene) (PTFE), tetrafluoroethylene-perfluoroalkylvinyl ether copolymer (PFA), tetrafluoroethylene-hexafluoropropylene copolymer (FEP), poly(chlorotrifluoroethylene) (PCTFE), poly(vinylidene fluoride) (PVDF), poly(vinyl fluoride) (PVF) as well as tetrafluoroethylene-ethylene copolymer (PETFE) or blends of the foregoing. Furthermore, in order to improve the heat resistance and adhesiveness in this embodiment of the present invention, there is no problem with mixing ceramic or carbon powders or the like with the resin used to form fluororesin adhesive layer 13.

Fluororesin layer 14 is preferably formed from at least one fluororesin selected from the group consisting of poly(tetrafluoroethylene) (PTFE), tetrafluoroethylene-perfluoroalkylvinyl ether copolymer (PFA), poly(chlorotrifluoroethylene) (PCTFE), poly(vinylidene fluoride) (PVDF), poly(vinyl fluoride) (PVF)), tetrafluoroethylene-hexafluoropropylene copolymer (FEP) or tetrafluoroethylene-ethylene copolymer (PETFE). Furthermore, there is no problem with having irregularities in the surface of fluororesin layer 14. If there are irregularities in the surface, this will further improve the sliding characteristics.

Improved sliding characteristics layer 15 is preferably formed from an agent for improving the sliding characteristics such as a chemical substance as is shown below in the generic formulas (1) through (6), or from poly(ethylene glycol), a polycation, a polyanion or the like. Furthermore, the use of suitable combinations thereof is also acceptable.
R¹O(CH₂CH₂O)ₙH (1)
(wherein, R¹ is a C8-C22 hydrocarbon group and n is 1-5), or by generic formula (2) below:
R²COO(CH₂CH₂O)ₙH (2)
(wherein, R² is a C7-C21 hydrocarbon group and n is 1-3), or by generic formula (3) below:
R³O(CH₂CH(CH₃)O)ₘH (3)
(wherein, R³ is a C8-C22 hydrocarbon group and m is 1-5), or by generic formula (4) below:
R⁴COO(CH₂CH(CH₃)O)ₘH (4)
(wherein, R⁴ is a C7-C21 hydrocarbon group and m is 1-3), or by generic formula (5) below:
R⁵O(CH₂CH(CH₃)O)ₛ(CH₂CH₂O)ₜH (5)
(wherein, R⁵ is a C8-C22 hydrocarbon group and s and t are 1-5), or by generic formula (6) below:
R⁶COO(CH₂CH(CH₃)O)ᵤ(CH₂CH₂O)ᵥH (6)
wherein, R⁶ is a C7-C21 hydrocarbon group and u and v are 1-3).

Furthermore, more preferred among these are polyoxyethylene (5 moles) sorbitan monooleic acid ester, polyoxyethylene (4 moles) sorbitan monostearic acid ester, polyoxyethylene (4 moles) lauryl ether, polyoxyethylene (4 moles) stearyl ether, diethylene glycol monolauryl ether, poly[oxyethylene(3 moles)oxypropylene(2 moles)] lauryl ether, diethylene glycol monolauric acid ester, diethylene glycol monostearyl ether, poly[oxyethylene(3 moles)oxypropylene(3 moles)] myristic acid ester, diethylene glycol stearic acid ester, propylene glycol monolauric acid ester, propylene glycol monostearic acid ester, propylene glycol monolauryl ether, poly(ethylene glycol) monooleate, polyoxyethylene oleyl ether and the like, and especially preferred is where n is 2 as in poly(ethylene glycol) monooleate <CH₃(CH₂)₇CH═CH(CH₂)₇COO(CH₂CH₂O)₂H> (also referred to as diethylene glycol monooleate) or poly(ethylene glycol) oleyl ether <CH₃(CH₂)₇CH═CH(CH₂)₈O(CH₂CH₂O)₂H>. Furthermore, the use of suitable combinations of such agents for improving the sliding characteristics is also acceptable.

Moreover, in this embodiment of the present invention, examples of polycations can include quaternized polyacrylamide, or polyvinylpyrrolidine, polyvinylamine, polyallylamine, polyethyleneimine, polyvinylvinylidene, Nafion (registered trademark) that possesses a quaternized end group, poly(dimethyldiallylammonium chloride), as well as synthetic and natural polymers of electrolytes that possess quaternized nitrogen-containing end groups and the like. In addition, the use of suitable combinations of such polycations is also acceptable. Additionally, in this embodiment of the present invention, examples of polyanions can include poly(ethylene sulfonate), poly(styrene sulfonate), Nafion, perfluoroacid ionomers and the like. Furthermore, the use of suitable combinations of such polyanions is also acceptable.

Moreover, in the above mentioned agent for improving the sliding characteristics, some or all of the hydrogen atoms can be substituted by fluorine atoms. In addition, in this embodiment of the present invention, a thickness of 0.1-2 μm for the improved sliding characteristics layer is preferred.

Additionally, in this embodiment of the present invention, the component with the improved sliding characteristics layer removed is referred to as the intermediate wire (substrate). Moreover, this intermediate wire can be manufactured according to the subject matter described in Japanese patent application 2005-137145.

At the same time, medical wire with improved sliding characteristics 10 according to this embodiment of the present invention is provided with metal wire 11, intermediate resin layer 12, fluororesin adhesive layer 13, fluororesin layer 14 and improved sliding characteristics layer 15, but the medical wire with improved sliding characteristics in accordance with the present invention is not limited to such a structure, and it is acceptable for at least one from among intermediate resin layer 12, fluororesin adhesive layer 13 to be omitted from medical wire with improved sliding characteristics 10, or to introduce additional layers into medical wire with improved sliding characteristics 10. In addition, it is acceptable to replace the fluororesin layer with an olefin resin layer, an ester resin layer, an acrylate resin layer, a polyurethane resin layer or a polyamide resin layer, as well as a layer of blends of such resins or the like.

(2) Manufacturing Method for a Medical Wire with Improved Sliding Characteristics

Medical wire with improved sliding characteristics 10 according to the present invention is principally manufactured through a dipping operation, a washing operation and a drying operation.

In the dipping operation, after the intermediate wire is immersed for a prescribed length of time in a solution of an agent for improving the sliding characteristics that has been adjusted to a given concentration at a given temperature, this intermediate wire is withdrawn from the solution. Additionally, in this embodiment of the present invention, the component after the intermediate wire is withdrawn from the solution of an agent for improving the sliding characteristics is referred to as the intermediate wire coated with an agent for improving the sliding characteristics. Moreover, this embodiment of the present invention is not limited to a particular immersion time, and it is acceptable for the intermediate wire to be withdrawn immediately after it has been immersed.

In the washing operation, the intermediate wire coated with an agent for improving the sliding characteristics is thoroughly washed with water. Furthermore, there is no particular limitation regarding the temperature of the water used for this operation, but the use of water at a temperature ≦30° C. is preferred.

In the drying operation, the intermediate wire coated with an agent for improving the sliding characteristics that has been washed with water in the washing operation is dried at room temperature.

[Medical Catheter with Improved Sliding Characteristics]

(1) Configuration of the Medical Catheter with Improved Sliding Characteristics

As described in FIG. 2, medical catheter with improved sliding characteristics 20 in this embodiment of the present invention is provided with polyimide tube 22 that includes fluororesins 23, 24 on its inner and outer surfaces (referred to below simply as PI tube), and with improved sliding characteristics layer 25 that coats the inner surface of PI tube 22.

PI tube 22 is formed from polyimide resin that includes fluororesins 23, 24 on its inner and outer surfaces as mentioned above. Moreover, such a PI tube 22 can be manufactured according to the subject matter described in Japanese published unexamined application No. 2005-205183. This polyimide resin is prepared from the poly(amic acid) obtained by the reaction of an aromatic tetracarboxylic acid dianhydride with an aromatic diamine in a polar organic solvent, followed by thermal transformation or chemical treatment. Representative examples of aromatic tetracarboxylic acid dianhydrides can include 3,3′,4,4′-benzophenonetetracarboxylic acid dianhydride, pyromellitic acid dianhydride, 2,3,3′,4-biphenyltetracarboxylic acid dianhydride, 3,3′,4,4′-biphenyltetracarboxylic acid dianhydride (BPDA), 1,2,5,6-naphthalenetetracarboxylic acid dianhydride, 1,4,5,8-naphthalenetetracarboxylic acid dianhydride, 2,3,6,7-naphthalenetetracarboxylic acid dianhydride, 2,2′-bis-(3,4-dicarboxyphenyl)propane dianhydride, perylene-3,4,9,10-tetracarboxylic acid dianhydride, bis-(3,4-dicarboxyphenyl) ether dianhydride, bis-(3,4-dicarboxyphenyl) sulfone dianhydride and the like. Additionally, representative examples of aromatic diamines can include 4,4′-diamino diphenyl ether, p-phenylenediamine (pPDA), m-phenylenediamine, 1,5-diaminonaphthalene, 3,3′-dichlorobenzidine, 3,3′-diaminodiphenylmethane, 4,4′-diaminodiphenylmethane, 3,3′-dimethyl-4,4′-biphenyldiamine, 4,4′-diaminodiphenylsulfide, 3,3′-diaminodiphenyl sulfone, benzidine, 3,3′-dimethylbenzidine, 4,4′-diaminophenyl sulfone, 4,4′-diaminodiphenylpropane, m-xylylenediamine, hexamethylenediamine, diaminopropyltetramethylene, 3-methylheptamethylenediamine and the like. In addition, these aromatic tetracarboxylic acid dianhydrides and aromatic diamines can be used singly or in mixtures. Moreover, fluororesins 23, 24 are preferably at least one fluororesin selected from the group consisting of poly(tetrafluoroethylene) (PTFE), tetrafluoroethylene-perfluoroalkylvinyl ether copolymer (PFA), poly(chlorotrifluoroethylene) (PCTFE), poly(vinylidene fluoride) (PVDF), poly(vinyl fluoride) (PVF)), tetrafluoroethylene-hexafluoropropylene copolymer (FEP) and tetrafluoroethylene-ethylene copolymer (PETFE).

Improved sliding characteristics layer 25 is formed in the same way as for the agent for improving the sliding characteristics that forms improved sliding characteristics layer 15 of medical wire with improved sliding characteristics 10.

Additionally, in this embodiment of the present invention, the component after removal of the improved sliding characteristics layer is referred to as the intermediate catheter (substrate).

At the same time, medical catheter with improved sliding characteristics 20 in this embodiment of the present invention is provided with PI tube 22 and improved sliding characteristics layer 25, but the medical catheter with improved sliding characteristics according to the present invention is not limited to such a structure, and it is acceptable for the PI tube to include fluororesin on the inner surface only, or for additional layers to be introduced into medical catheter with improved sliding characteristics 20. In addition, it is also acceptable for irregularities to be formed in the surface. Moreover, it is also acceptable for at least one of the polyimide resin and the fluororesin to be substituted with an olefin resin, an ester resin, an acrylate resin layer, a polyurethane resin or a polyamide resin, as well as blends of such resins or the like.

(2) Manufacturing Method for a Medical Catheter with Improved Sliding Characteristics

The medical catheter with improved sliding characteristics according to the present invention is principally manufactured through an operation for blocking one end, an operation for pouring in a solution of an agent for improving the sliding characteristics, an operation for sealing in the solution of the agent for improving the sliding characteristics, an operation for draining the solution of the agent for improving the sliding characteristics, a washing operation and a drying operation.

In the operation for blocking one end, one end of the intermediate catheter is blocked. Furthermore, methods for blocking can include blocking methods such as tying a knot in one end of the intermediate catheter, plugging up the opening at one end of the intermediate catheter, taping the opening at one end of the intermediate catheter and the like.

In the operation for pouring in a solution of an agent for improving the sliding characteristics, the solution of an agent for improving the sliding characteristics is poured into borehole 21 of the intermediate catheter through the open end of the intermediate catheter using a syringe or the like.

In the operation for sealing in the solution of the agent for improving the sliding characteristics, the open end of the intermediate catheter is blocked, and after the solution of the agent for improving the sliding characteristics has been sealed within the borehole of the intermediate catheter for the given period of time, this intermediate catheter is allowed to stand at a given temperature maintained in a constant temperature bath. Further examples of this blocking method can include the same blocking methods as in the operation for blocking one end such as tying a knot in one end of the intermediate catheter, plugging up the opening at one end of the intermediate catheter, taping the opening at one end of the intermediate catheter and the like. In addition, this embodiment of the present invention is not limited to a particular time period for the sealing in of the solution of an agent for improving the sliding characteristics, so that after the solution of an agent for improving the sliding characteristics has been introduced into borehole 21 of the intermediate catheter, it is acceptable for the solution of the agent for improving the sliding characteristics to be drained immediately (in other words, it is acceptable to abbreviate the present operation).

In the operation for draining the solution of the agent for improving the sliding characteristics, one end of the intermediate catheter is opened, and the solution of the agent for improving the sliding characteristics is drained from within borehole 21 of the intermediate catheter. Furthermore, examples of this method for opening can include cutting one end of the intermediate catheter, pulling out the plug in the opening at one end of the intermediate catheter, tearing off the tape or the like used to block the opening at one end of the intermediate catheter, and the like. Furthermore, in this embodiment of the present invention, the intermediate catheter from which the solution of the agent for improving the sliding characteristics has been drained is referred to as the intermediate catheter coated with the agent for improving the sliding characteristics.

In the washing operation, with the openings at both ends of the intermediate wire coated with the agent for improving the sliding characteristics being opened, water is poured into borehole 21 of the intermediate wire coated with the agent for improving the sliding characteristics using a syringe or the like, and the inner walls of the borehole are washed thoroughly with water while at the same time the given borehole diameter is preserved. Furthermore, there is no particular limitation regarding the temperature of the water used for this operation, but the use of water at a temperature ≦30° C. is preferred.

In the drying operation, the intermediate catheter coated with the agent for improving the sliding characteristics that has been washed with water in the washing operation is dried at room temperature.

[Measuring Various Physical Properties of the Medical Wire with Improved Sliding Characteristics and the Medical Catheter with Improved Sliding Characteristics]

Here, methods are explained for measuring the values of the contact angle and of the frictional resistance for medical wire with improved sliding characteristics 10.

(1) Method for Measuring the Contact Angle for an Agent for Improving the Sliding Characteristics

In this embodiment of the present invention, a sheet sample that includes a PTFE surface is immersed in a solution of an agent for improving the sliding characteristics that is adjusted to a given temperature, and after standing for 30 min, it is washed thoroughly with water and dried at ambient temperature to produce the sample for use in measuring the contact angle. Then, in this embodiment of the present invention, the contact angle for purified water at 23° C. is measured using a Face CA-Z analyzer

(Kyowa Interface Science Co.).

(2) Method for Measuring the Value of the Frictional Resistance for Medical Wire with Improved Sliding Characteristics

The frictional resistance for medical wire with improved sliding characteristics 10 was measured using frictional resistance measuring apparatus 30 as shown in FIG. 3.

When using this frictional resistance measuring apparatus 30, first polyurethane resin tube 32 (ID 2.5 mm, OD 4.0 mm, length 200 mm) was fastened with an adhesive to metal cylindrical jig 31 (dia. 90 mm). Then, this metal cylindrical jig 31 was mounted onto the stationary side chuck 41 of tensile tester 40. Next, medical wire with improved sliding characteristics 10 in this embodiment of the present invention was introduced into polyurethane tube 32, and one end of this medical wire with improved sliding characteristics 10 was attached to clip 42 of tensile tester 40. Then, under these conditions, medical wire with improved sliding characteristics 10 was pulled in the vertical upwards direction (Da) at a rate of 50 mm/min, and the load at this time was measured using load cell 43. In other words, in the present measurement method, the frictional resistance between medical wire with improved sliding characteristics 10 and polyurethane resin tube 32 was measured. Furthermore, at this time it was found that the frictional resistance was smaller at lower tensile loads. Additionally, in this embodiment of the present invention, when the measurement is being made, the frictional resistance value could be measured from any desired positions of medical wire with improved sliding characteristics 10 along a length of 50 mm, and these frictional resistance values were recorded on chart paper. Moreover, in this embodiment of the present invention, the measurement can be repeated multiple times and from the multiple values obtained for the frictional resistance an average can be determined that can be taken as the final value for the frictional resistance.

In addition, in order to determine a value for the frictional resistance in water for. medical wire with improved sliding characteristics 10, it is acceptable to fill the borehole of polyurethane tube 32 with water

(3) Method for Measuring the Value of the Frictional Resistance for Medical Catheter with Improved Sliding Characteristics

The value of the frictional resistance for medical catheter with improved sliding characteristics 20 can be measured in the same manner as was used in measuring the frictional resistance for medical wire with improved sliding characteristics, and this measurement was made using frictional resistance measuring apparatus 30 as shown in FIG. 3. However, in order to improve the sensitivity, a metal cylindrical jig 31 with a smaller diameter was used and the tensile rate was increased.

When using this frictional resistance measuring apparatus 30, first a stainless steel wire coated with fluororesin (dia. 0.35 mm, referred to as the reference wire) was introduced into borehole 21 of medical catheter with improved sliding characteristics 20, and 25 mm portions of both ends of medical catheter with improved sliding characteristics 20 were maintained straight respectively while the catheter was wound four times around the circumference of metal cylindrical jig 31 having a diameter of 67 mm, and was fastened there with adhesive tape. Then, this metal cylindrical jig 31 was mounted onto the stationary side chuck 41 of tensile tester 40. Then, under these conditions, the reference wire was pulled in the vertical upwards direction (Da) at a rate of 200 mm/min, and the load at this time was measured using load cell 43. In other words, in the present measurement method, the frictional resistance between the inner surface of medical catheter with improved sliding characteristics 20 and the fluororesin was measured. Furthermore, at this time it was found that the frictional resistance was smaller at lower tensile loads. In addition, in this embodiment of the present invention, the values of the tensile load over a tensile distance of 100 mm were recorded on a chart. Additionally, in this embodiment of the present invention, the measurement can be repeated multiple times and from the multiple values obtained for the frictional resistance an average can be determined as the final value for the frictional resistance.

Furthermore, in order to determine a value for the frictional resistance in water for medical catheter with improved sliding characteristics 20, it is acceptable to fill the interior of borehole 21 of medical catheter with improved sliding characteristics 20 with water.

(4) Method for Measuring the Thickness of an Agent for Improving the Sliding Characteristics

The thickness of an agent for improving the sliding characteristics was measured with the use of an ellipsometer (DHA-XAVW/S6, Mizojiri Optical Co., Ltd.).

### WORKING EXAMPLES

A number of working examples based on medical wire with improved sliding characteristics 10 and the medical catheter with improved sliding characteristics 20 in this embodiments of the present invention are shown below.

### Working Example 1

Into a 1.0 wt % aqueous solution of poly(ethylene glycol monooleate) (PGO) (with two oxyethylene repeating units) (referred to below as PGO aqueous solution) adjusted to 60° C. was immersed a polyimide sheet that included PTFE on both sides, and after standing for 30 min, this sheet was thoroughly washed with water and then dried at ambient temperature. Next, the contact angles between both sides of this sheet sample and purified water were measured at 23° C. using a contact-angle analyzer (Face CA-Z, Kyowa Interface Science Co.). The results are found in Table 1.

### Working Example 2

Into a 1.0 wt % aqueous solution of poly(oxyethylene oleyl ether) (POO) (with two oxyethylene repeating units) (referred to below as POO aqueous solution) adjusted to 60° C. was immersed a polyimide sheet that included PTFE on both sides, and after standing for 30 min, this sheet was thoroughly washed with water and then dried at ambient temperature. Next, the contact angles between both sides of this sheet sample and purified water were measured at 23° C. using a contact-angle analyzer (Face CA-Z, Kyowa Interface Science Co.). The results are found in Table 1.

### Working Example 3

A PTFE sheet was immersed in the PGO aqueous solution adjusted to 60° C. of Working Example 1, and after standing for 30 min this sheet was thoroughly washed with water and then dried at ambient temperature. Next, the contact angle with purified water was measured at 23° C. using a contact-angle analyzer (Face CA-Z, Kyowa Interface Science Co.). The results are found in Table 1.

### Working Example 4

A PTFE sheet was immersed in the POO aqueous solution adjusted to 60° C. of Working Example 2, and after standing for 30 min this sheet was thoroughly washed with water and then dried at ambient temperature. Next, the contact angle with purified water was measured at 23° C. using a contact-angle analyzer (Face CA-Z, Kyowa Interface Science Co.). The results are found in Table 1.

### Working Example 5

Two stainless steel wires having a polyurethane layer, a PTFE adhesive layer and a PTFE layer (referred to below as the first intermediate wire) were prepared, and these were immersed respectively into a 1.0 wt % and a 5.0 wt % aqueous solution of PGO adjusted to 60° C., and after standing for 30 min these wires were thoroughly washed with water and then dried at ambient temperature. Then, after this, the excess attached PGO on the medical wire with improved sliding characteristics was trimmed away, so that the final PGO layer thickness obtained was within the range 0.1-2 μm. Furthermore, in the first intermediate wire, a polyurethane layer is formed on the outer surface of the stainless steel wire, a PTFE adhesive layer is formed on the outer surface of the polyurethane layer, and a PTFE layer is formed on the outer surface of the PTFE adhesive layer. In other words, the outermost layer of the first intermediate wire is a PTFE layer.

The frictional resistance of this medical wire with improved sliding characteristics in the dry state and the wet state was measured according to the abovementioned measurement method. The results are found in Table 2.

### Working Example 6

Two stainless steel wires equipped with a PTFE layer (referred to below as the second intermediate wire) were prepared, and these were immersed respectively into a 1.0 wt % and a 5.0 wt % aqueous solution of PGO adjusted to 60° C., and after standing for 30 min these wires were thoroughly washed with water and then dried at ambient temperature. Then, after this, the excess attached PGO on the medical wire with improved sliding characteristics was trimmed away, so that the final PGO layer thickness obtained was within the range 0. 1-2 μm. Furthermore, in the second intermediate wire, a PTFE layer is formed on the outer surface of the stainless steel wire. In other words, the outermost layer of the second intermediate wire is a PTFE layer.

The frictional resistance of this medical wire with improved sliding characteristics in the dry state and the wet state was measured according to the above mentioned measurement method. The results are found in Table 3.

### Working Example 7

Two polyimide tubes (acid dianhydride: BPDA; diamine: pPDA) (length: 900 mm; ID: 0.5 mm) that included PTFE on their inner surfaces and were closed at one end were prepared, and respectively a 1.0 wt % and a 5.0 wt % aqueous solution of PGO adjusted to 60° C. was introduced into the boreholes using a syringe, and the boreholes of these polyimide tubes were filled with the PGO aqueous solutions and these were sealed in. In this state, the tubes were placed into a 60° C. water bath, and this temperature was maintained as the tubes stood for 30 min. Afterward, the PGO aqueous solutions were poured out of the boreholes, then a syringe was used to introduce sufficient water into the tubes and the inner surfaces of the tubes were washed thoroughly with water.

The frictional resistance in water of the inner surface of these medical catheters with improved sliding characteristics was measured according to the abovementioned measurement method. The results are found in Table 4. Furthermore, the initial measurement of frictional resistance and the 100^{th} measurement of the frictional resistance after carrying out 100 repeated cycles of the same measurement are shown in Table 4. In addition, the data for an unmodified PTFE tube is shown side by side in Table 4 as a comparison example.

### Working Example 8

A polyimide tube (acid dianhydride: BPDA; diamine: pPDA) (length: 900 mm; ID: 0.5 mm) that included PTFE on its inner surface and was closed at one end was prepared, and a 1.0 wt % aqueous solution of PGO adjusted to 60° C. was introduced into the borehole using a syringe, and the borehole of this polyimide tube was filled with the PGO aqueous solution and this was sealed in. In this state, the tube was placed into a 60° C. water bath, and this temperature was maintained as the tube stood for 30 min. Afterward, the PGO aqueous solution was poured out of the borehole, then a syringe was used to introduce sufficient water into the tube and the inner surface of the tube was washed thoroughly with water. A medical catheter with improved sliding characteristics manufactured in this way is referred to below as a PGO-coating catheter.

Additionally, after a polyimide tube (acid dianhydride: BPDA; diamine: pPDA) (length: 900 mm; ID: 0.5 mm) that included PTFE on its inner surface was closed at one end, a 1.0 wt % aqueous solution of POO adjusted to 60° C. was introduced into the borehole using a syringe, and the borehole of this polyimide tube was filled with the POO aqueous solution and this was sealed in. In this state, the tube was placed into a 60° C. water bath, and this temperature was maintained as the tube stood for 30 min. Afterward, the POO aqueous solution was poured out of the borehole, then a syringe was used to introduce sufficient water into the tube and the inner surface of the tube was washed thoroughly with water. A medical catheter with improved sliding characteristics manufactured in this way is referred to below as a POO-coating catheter.

Then, after the respective boreholes of the PGO-coating catheter, the POO-coating catheter and a polyimide tube including PTFE on its inner surfaces are filled with water, fluororesin-coated stainless steel wire (dia. 0.35 mm) (referred to below as the reference wire) is introduced into the respective boreholes, and the respective catheters are installed in identical blood vessel models (supports that can be formed into realistic human arterial vessels for the catheters). Furthermore, in the present working example the length of the catheters is 1 m. Eight test subjects then pulled, pushed and turned the reference wire, and gave a “1” for the most superior sliding characteristics, a “2” for the next most superior sliding characteristics, and gave a “3” for inferior sliding characteristics. The results are found in Table 5.

### Working Example 9

Two PTFE tubes (length: 900 mm; ID: 0.5 mm) that were closed at one end were prepared, and into the boreholes of these were poured respectively a 1.0 wt % and a 5.0 wt % aqueous solution of PGO adjusted to 60° C. using a syringe, and the boreholes of these PTFE tubes were filled with the PGO aqueous solution and this was sealed in. In this state, the tubes were placed into a 60° C. water bath, and this temperature was maintained as the tubes stood for 30 min. Afterward, the PGO aqueous solution was poured out of the boreholes, then a syringe was used to introduce sufficient water into the tubes and the inner surfaces of the tubes were washed thoroughly with water.

The frictional resistance of these medical catheters with improved sliding characteristics in the dry condition and the wet condition were measured according to the abovementioned measurement method. The results are found in Table 6.

TABLE 1

| | | | No |
|---|---|---|---|
| | PGO | POO | surface improvement agent |
| PI containing PTFE (obverse | 57 | 50 | 110 |
| surface) | | | |
| PI containing PTFE (reverse | 62 | 57 | 108 |
| surface) | | | |
| PTFE | 55 | 52 | 108 |

(Units: °)

TABLE 2

| Outermost layer | Dry state | Wet state |
|---|---|---|
| PTFE | 100 | 115 |
| PGO (1.0 wt %) | 75 | 58 |
| PGO (5.0 wt %) | 74 | 59 |

(Units: mN)

TABLE 3

| Outermost layer | Dry state | Wet state |
|---|---|---|
| PTFE | 10 | 8.2 |
| PGO (1.0 wt %) | 6.2 | 5 |
| PGO (5.0 wt %) | 5.8 | 5 |

(Units: mN)

TABLE 4

| | PGO (1.0 wt %) | PGO (5.0 wt %) | PTFE |
|---|---|---|---|
| Initial run | 62 | 64 | 80 |
| After 100 experimental runs | 61 | 59 | 80 |

(Units: mN)

TABLE 5

| Test subject | PGO | POO | PTFE |
|---|---|---|---|
| A | 1 | 2 | 3 |
| B | 1 | 3 | 2 |
| C | 1 | 2 | 3 |
| D | 1 | 2 | 3 |
| E | 1 | 2 | 3 |
| F | 1 | 2 | 3 |
| G | 1 | 2 | 3 |
| H | 1 | 2 | 3 |

TABLE 6

| Inner surface | Dry state | Wet state |
|---|---|---|
| PTFE | 80 | 78 |
| PGO (1.0 wt %) | 58 | 56 |
| PGO (5.0 wt %) | 54 | 57 |

(Units: mN)

[Results]

As is clear from Table 1, PGO and POO as the agents for improved sliding characteristics are effective at making the PTFE surface hydrophilic.

Additionally, as is clear from Tables 2, 3, 5 and 6, medical wires and catheters with improved sliding characteristics due to PGO and POO exhibit sliding characteristics that are superior to those of medical wires and catheters that possess PTFE surfaces in both the dry state and the wet state.

Moreover, as is clear from Table 4, a medical wire with improved sliding characteristics due to PGO exhibits sustained sliding characteristics from experiments involving 100 repeated cycles, and exhibits superior durability when used repeatedly.

### INDUSTRIAL APPLICABILITY

A medical device for insertion into the human body in accordance with the present invention exhibits sliding characteristics in both the dry state and in the wet state that are superior to those in a medical device for insertion into the human body that possesses a fluororesin surface, and furthermore the present medical device has superior durability, and possesses the characteristic that the extent of degradation due to repeated use will be extremely small, and is useful as a medical catheter or medical wire or the like.

1. A medical device for insertion into the human body, comprising:
a substrate having a pre-determined shape and a hydrophobic surface on at least one portion, and
an agent for improving the sliding characteristics having a hydrophilic block and a hydrophobic block, and coating said hydrophobic surface.

2. The medical device for insertion into the human body according to claim 1, wherein said hydrophobic surface is a fluororesin surface.

3. The medical device for insertion into the human body according to claim 1, wherein said agent for improving the sliding characteristics possesses a thickness of 0.1-2 μm.

4. The medical device for insertion into the human body according to claim 1, wherein said hydrophilic block is a polyoxyethylene group.

5. The medical device for insertion into the human body according to claim 4, wherein the number of the repeating unit of said polyoxyethylene group comprises is 1 to 5.

6. The medical device for insertion into the human body according to claim 1, wherein said hydrophobic block is a hydrocarbon group.

7. The medical device for insertion into the human body according to claim 6, wherein said hydrocarbon group is selected from the group consisting of oleyl group, lauryl group and stearyl group.

8. The medical device for insertion into the human body according to claim 1, wherein said agent for improving the sliding characteristics is represented by generic formula (1) below:
R¹O(CH₂CH₂O)ₙH (1)
(wherein, R¹ is a C8-C22 hydrocarbon group and n is 1-5), or by generic formula (2) below:
R²COO(CH₂CH₂O)ₙH (2)
(wherein, R² is a C7-C21 hydrocarbon group and n is 1-3), or by generic formula (3) below:
R³O(CH₂CH(CH₃)O)ₘH (3)
(wherein, R³ is a C8-C22 hydrocarbon group and m is 1-5), or by generic formula (4) below:
R⁴COO(CH₂CH(CH₃)O)ₘH (4)
(wherein, R⁴ is a C7-C21 hydrocarbon group and m is 1-3), or by generic formula (5) below:
R⁵O(CH₂CH(CH₃)O)ₛ(CH₂CH₂O)ₜH (5)
(wherein, R⁵ is a C8-C22 hydrocarbon group and s and t are 1-5), or by generic formula (6) below:
R⁶COO(CH₂CH(CH₃)O)ᵤ(CH₂CH₂O)ᵥH (6)
(wherein, R⁶ is a C7-C21 hydrocarbon group and u and v are 1-3).

9. The medical device for insertion into the human body according to claim 2, wherein said hydrophobic block is a fluorine-containing hydrocarbon group.

10. The medical device for insertion into the human body according to claim 1, wherein said substrate is a medical wire.

11. The medical device for insertion into the human body according to claim 1, wherein said substrate is a catheter.

12. The medical device for insertion into the human body according to claim 11, wherein said hydrophobic surface exists on at least one of the inner surface and outer surface of said catheter.

13. A method for manufacturing the medical device for insertion into the human body according to claim 1, comprising
applying a solution of said agent for improving the sliding characteristics to said hydrophobic surface of said substrate, and
drying said substrate coated with said agent for improving the sliding characteristics.

14. The method for manufacturing the medical device for insertion into the human body according to claim 13, wherein said solution contains 0.1-5 wt % of said agent for improving the sliding characteristics.

15. The method for manufacturing the medical device for insertion into the human body according to claim 14, wherein said agent for improving the sliding characteristics is poly(ethylene glycol) monooleate and said solution is maintained at 60±5° C.

16. The medical device for insertion into the human body according to claim 2, wherein said hydrophilic block is a polyoxyethylene group.

17. The medical device for insertion into the human body according to claim 2, wherein said hydrophobic block is a hydrocarbon group.
